(19) United States
(12) Patent Application Publication
(10) Pub. No.: US 2025/0191738 A1
(43) Pub. Date: Jun. 12, 2025

(54) TRIAGE AND RESOURCE OPTIMISATION SYSTEM FOR COMMUNITY-BASED PALLIATIVE CARE

(71) Applicant: University College Cork, Cork City (IE)

(72) Inventors: Ciara Heavin, Cork (IE); Armagan Tarim, Co. Cork (IE); Fiona Kiely, Cork (IE)

(73) Assignee: University College Cork, Cork City (IE)

(21) Appl. No.: 18/974,043

(22) Filed: Dec. 9, 2024

Related U.S. Application Data
(60) Provisional application No. 63/607,911, filed on Dec. 8, 2023.

(51) Int. Cl. G16H 40/20; G16H 10/60; G16H 80/00

(52) U.S. Cl. CPC G16H 40/20; G16H 10/60; G16H 80/00

(57) ABSTRACT

The invention provides a computer implemented community care triage and resource optimisation method and system including a predictive analytics component, a prescriptive analytics component, and a user interface component. The predictive analytics component receives a plurality of inputs relating to biographical and sociodemographic information and health status of a plurality of patients and outputs predictions for a plurality of future healthcare needs and health attributes. The prescriptive analytics component receives a plurality of organisational data inputs related to patients' information, healthcare workers information and medical equipment information for a given period and combined with results from the predictive analytics component, and outputs an optimal resource allocation for the given period following one or more guidelines. The user interface component provides a dynamic schedule which incorporates the outputted optimal resource allocation for the given period following one or more guidelines using one or more interactive and visual representations.

This application claims the benefit of U.S. Provisional Patent Application No. 63/607,911, filed 8 Dec. 2023, the specification of which is hereby incorporated herein by reference.

### BACKGROUND OF THE INVENTION

### Field of the Invention

At least one embodiment of the invention relates to healthcare informatics. More particularly, the disclosure relates to the use of machine learning (ML), artificial intelligence (AI) and operations research (OR) in a health care environment.

### Description of Related Art

The number of people in need of palliative care is increasing across the world, primarily due to increased life expectancy, advances in medicine, and improving standards of living. Home Healthcare (HHC) and community healthcare (CHC) are becoming some of the most important components of healthcare systems internationally. Factors such as ageing populations, chronic diseases, and the insufficient capacity of acute hospitals contribute to the increasing need for HHC and CHC. Healthcare workers have had a difficult time managing a wide range of patient, clinical health, and operational data, forever searching for ways to improve the delivery of care in practice management. There are many indicators pointing to an increase in anticipated demand on these service in the near future. As the relative size of the labour force shrinks there will be less healthcare budget per older person. Hence it is not just a question of upscaling existing services but of disrupting service allocation and delivery to do things more efficiently and effectively.

There exist systems for HHC and CHC palliative care management which incorporate relatively low levels of analytical capabilities for patient assessment and resource allocation. These capabilities include enabling auto-scheduling of single appointments, wherein a manager can specify constraints (e.g., skills of a healthcare worker) and the system returns suggestions for suitable times and a carer to be scheduled. Other systems enable carers to utilise analytics to automate some of the triaging tasks but there is no integration of a caseload management and so it needs to be manually set. These systems do not address the allocation of HHC and CHC services which combine triaging with daily caseload management and so they do not predict or respond to the urgency of need of patients.

There exist approaches to the HHC and CHC resource optimisation problem within the context of palliative care in the community. These rely on diverse metaheuristic approaches including tabu search and an inexact Benders method implementing a separate solution comprising rostering and scheduling components to interrogate this problem. These approaches are limited by the assumption that service operation parameters such as patient service time and healthcare worker travel times are precisely known without uncertainty. As a result, these existing approaches do not address the HHC and CHC resource optimisation problem when various operation parameters are non-deterministic and hence have a range of possible values. This is the case with real world HHC and CHC operation parameters, and so these approaches do not specifically address real world HHC and CHC management.

There is therefore a need for a system for community based palliative care which optimises resource use which combines triaging of patients with complex needs that are subject to change and the allocation of a daily caseload that is achievable for healthcare workers scheduling and delivering care. There is an additional need for such a system which includes uncertainty in operation parameters.

### BRIEF SUMMARY OF THE INVENTION

At least one embodiment of the invention relates to a community care triage and resource optimisation system, enabling specialized healthcare in the community, which provides at least one or more of the following benefits:
maximise the number of patients cared for in a fair way;
improve healthcare worker (e.g. Clinical Nurse Specialist) support by providing them with an optimum and transparent plan to undertake their roles;
improve understanding the changing health and social needs of patients receiving care in the community;
get the right information to the right healthcare workers at the right time to support decision making to reduce uncertainty in the delivery of care;
improve the wellbeing of people with life-limiting illnesses, as well as that of their families;
optimise the routes taken by healthcare workers e.g. clinical nurse specialists with the aim of delivering a more sustainable healthcare service; and
optimise Greenhouse Gas (GHG) emissions for the service for their day.

According to one or more embodiments of the invention there is provided, as set out in the appended claims, a community care resource optimisation system to improve the delivery of palliative care in a community setting.

In at least one embodiment of the invention there is provided a computer implemented community care patient triaging and resource optimisation system comprising a predictive analytics component (triage), a prescriptive analytics component (resource allocation), and a user interface component, wherein;
the predictive analytics component is configured to receive a plurality of inputs relating to the sociodemographic data and the health status of a plurality of patients using existing clinical guidelines and configured to output predictions for a plurality of future healthcare needs and health attributes;
the prescriptive analytics component receives a plurality of organisational data inputs related to patients information, healthcare workers information and medical equipment information for a given period (for example a single day) and combined with results from the predictive analytics component, and output an optimal resource allocation for the given period following one or more guidelines;
the user interface component configured to provides a dynamic daily caseload which incorporates the outputted list of prioritised patients and the optimal resource allocation for the given period following one or more guidelines using one or more interactive and visual representations.

It will be appreciated that one or more embodiments of the invention include a system for the optimisation of resource use for community based palliative care, wherein machine learning models and operations research are implemented. A plurality of input data including location, preference and availability data for patients and healthcare workers, in combination with historical traffic and service length distributions are used. The output of the system is a recommended operational plan consisting of caseloads and routes for the healthcare workers which optimally uses the available resources to attend to the most urgent patients, while balancing financial costs and greenhouse gas emissions. The system identifies a subset of prioritised patients (based on their features) who should receive an assessment today.

In at least one embodiment of the invention, the plurality of inputs relating to the biographic, sociodemographic, and health information of a plurality of patients can contain one or more of: patient age, degree of social support for patient, if the patient lives alone, if the patient lives in residential care, if the patient has a carer, and if the patient has a family member as a carer.

In at least one embodiment of the invention, the predictive analytics component uses machine learning to assess patient care needs.

In at least one embodiment of the invention, the plurality of inputs relating to the health status of a plurality of patients contain one or more of: patient diagnosis, presence of multimorbidity in patient, assessment of patient's breathing, assessment of patient's sleeping, assessment of patient's bowel movements, assessment of need for urgent crisis event planning.

In at least one embodiment of the invention, one or more of the plurality of inputs relating to the health status of a patient are provided directly by a healthcare worker based on their assessment of the patient.

In at least one embodiment of the invention, one or more of the plurality of inputs relating to the health status of a patient are provided directly by the patient based on their own assessment through a device connected to the internet. Suitably the device can be a smart phone device or electronic tablet device or similar electronic device.

In at least one embodiment of the invention, one or more of the plurality of inputs relating to the health status of a patient are provided indirectly by the patient, or automatically by pervasive or internet of things technology, such as wearable health monitors, remote patient monitoring systems, smart home health systems, implantable devices, telehealth platforms with integrated monitoring.

In at least one embodiment of the invention, the plurality of healthcare needs output by the predictive analytics component comprises a probability distribution to determine a need for implementing one or more of medical interventions, wherein the probability distribution is a mathematical function quantifies the likelihood of different outcomes based on the sociodemographic data and the health status of a patient.

In at least one embodiment of the invention, the plurality of health attributes output by the predictive analytics component includes a measure of the stability of a patient's condition.

In at least one embodiment of the invention, the prescriptive analytics component uses constraint/stochastic programming to allocate resources.

In at least one embodiment of the invention, the plurality of inputs related to patients contains one or more of their: location, availability, plurality of healthcare needs and health attributes output by the predictive analytics component.

In at least one embodiment of the invention, the plurality of organisational data inputs related to healthcare workers comprises one or more of their: location, availability, speciality/skillset.

In at least one embodiment of the invention, the plurality of organisational data inputs related to medical equipment contains its availability for use.

In at least one embodiment of the invention, the given period is a single working day.

In at least one embodiment of the invention, the one or more guidelines includes reducing total transport costs.

In at least one embodiment of the invention, the one or more guidelines includes reducing total transport greenhouse gas emissions.

In at least one embodiment of the invention, the plurality of interactive and visual representations comprising the dynamic schedule display data relates to one or more of the following: the stability of the plurality of patients' care plans and priorities, the available resources, the preferences of patients, the preference of healthcare workers, projections of estimated time of arrival, travel distance, and opportunity cost of reprioritising patients.

In at least one embodiment of the invention the daily operational plan further comprises a map overview of patient locations.

In at least one embodiment of the invention, the plurality of interactive and visual representations comprising the dynamic schedule includes a daily operational plan, wherein the plan comprises a schedule for each healthcare worker with details of which patients they will tend to, the order in which they will visit them, and recommended routes with which to travel between patient locations.

In at least one embodiment of the invention, the user can revise the daily operational plan or choose from one or more operational planes provided, based on the plurality of interactive and visual representations that comprises the dynamic schedule.

In at least one embodiment, the system further comprises a single sign-on SSO access component configured to authenticate users and provide secure access to the system.

In at least one embodiment, the system is implemented as a cloud-based web application, enabling access through a web browser over the internet.

In at least one embodiment, the system is implemented as an API, enabling programmatic access to the predictive analytics component, the prescriptive analytics component, and the user interface component through standardized application programming interfaces over a network.

In at least one embodiment, the system is implemented as a cross-platform application, compatible with multiple operating systems and devices, allowing users to access its functionality seamlessly across desktop, mobile, and tablet platforms.

In at least one embodiment, the system is implemented as a native mobile application, designed for installation on mobile devices, providing access to its functionality through an intuitive mobile interface optimized for touch-screen interaction.

In at least one embodiment, the system further comprises a preference component configured to manage healthcare worker and patient preferences.

In at least one embodiment, the preference component outputs preference constraints, and wherein the plurality of organisational data inputs related to patients' information and healthcare workers information, received by the prescriptive analytics component, comprises the preference constraints.

In at least one embodiment, the preference constraints comprise continuous care of a patient by at least one healthcare worker known to the patient.

In at least one embodiment, the system further comprises a re-scheduler component configured to update the dynamic schedule provided by the user interface component.

In at least one embodiment, the re-scheduler component is configured to process a plurality of real-time updated organisational data inputs related to at least one of patient's information, healthcare workers information and medical equipment information, which is combined with results from the predictive analytics component.

In at least one embodiment, the dynamic schedule is updated when the re-scheduler determines the current dynamic schedule is not feasible, wherein the determination is based on the real-time updated organisational data inputs.

In at least one embodiment, the system comprises a re-scheduler component configured to update the dynamic schedule provided by the user interface component, wherein the re-scheduler component is configured to:
(a) process a plurality of real-time updated organisational data inputs related to at least one of: patient's information, a given healthcare workers information and medical equipment information;
(b) combine the processed data of step (a) with the results of the predictive analytics component to determine the feasibility of the schedule of the given healthcare worker; and
(c) update the schedule for the given healthcare worker based on the determination of step (b).

In at least one embodiment, the system further comprises a reporting component configured to store and report historical daily operational plans and data related to outcomes due to implementation of the daily operational plans.

In at least one embodiment, the reporting component is configured to report one or more of the following:
performance of implementation of the historical daily operational plans; outcomes due to implementation of the historical daily operational plans; historical user activities;
trends in the historical daily operational plans; and
greenhouse gas emissions caused by implementation of the historical daily operational plans.

In at least one embodiment, the system further comprises an alert component configured to analyse the dynamic schedule for critical events and alert the user to the critical events via the user interface component, wherein the critical events comprise a risk of one or more of:
high-priority patients not being tended to in a given time period;
a patients scheduled service being missed a second time;
patients not being seen within obligatory service level agreement window; and
a reschedule event occurring which is significantly less optimal than the previous schedule.

In at least one embodiment there is provided a community care and resource optimisation system comprising a predictive analytics component and a prescriptive analytics component wherein;
the predictive analytics component is configured to receive a plurality of inputs relating to the health status of a plurality of patients, and configured to output predictions for a plurality of future healthcare needs;
the prescriptive analytics component receives a plurality of organisational data inputs related to at least one of patients information, healthcare workers information and medical equipment information for a given period and combined with results from the predictive analytics component, and output an optimal resource allocation for the given period.

In at least one embodiment, the is provided a method for community care triage and resource optimisation, comprising the steps of:
receiving, via a predictive analytics component, a plurality of inputs relating to the sociodemographic information and the health status of a plurality of patients, and outputting predictions for a plurality of future healthcare needs and health attributes;
receiving, via a prescriptive analytics component, a plurality of organizational data inputs related to at least one of patient information, healthcare worker information, and medical equipment information for a given period;
combining the organizational data inputs with the predictions from the predictive analytics component, and outputting an optimal resource allocation for the given period following one or more guidelines; and
providing, via a user interface component, a dynamic schedule incorporating the outputted optimal resource allocation for the given period, wherein the schedule is presented using one or more interactive and visual representations.

In at least one embodiment:
the plurality of inputs relating to the sociodemographic information of a plurality of patients comprises one or more of: patient age, degree of social support for the patient, if the patient lives alone, if the patient lives in residential care, if the patient has a carer, and if the patient has a family member as a carer;
the plurality of inputs relating to the health status of a plurality of patients comprises one or more of: patient diagnosis, presence of multimorbidity, assessment of the patient's breathing, assessment of the patient's sleeping, assessment of the patient's bowel movements, and assessment of the need for urgent crisis event planning;
one or more of the plurality of inputs relating to the health status of a patient are provided directly by a healthcare worker based on their assessment of the patient; and
the plurality of inputs related to patients contains one or more of their location, availability, plurality of healthcare needs, and health attributes output by the predictive analytics component, and the plurality of organizational data inputs related to healthcare workers comprises one or more of their location, availability, and speciality/skillset.

In at least one embodiment:
the predictive analytics component comprises a machine learning module adapted to assess patient care needs; and
the plurality of interactive and visual representations comprising the dynamic schedule includes a daily operational plan, wherein the plan comprises a schedule for each healthcare worker with details of which patients they will tend to, the order in which they will visit them, and recommended routes with which to travel between patient locations.

In at least one embodiment, there is a computer-implemented method for community care triage and resource optimisation, comprising the steps of:
receiving, via a predictive analytics component, a plurality of inputs relating to the health status of a plurality of patients, and outputting predictions for a plurality of future healthcare needs;
receiving, via a prescriptive analytics component, a plurality of organizational data inputs related to at least one of patient information, healthcare worker information, and medical equipment information for a given period;
combining the organizational data inputs with the predictions from the predictive analytics component; and
outputting an optimal resource allocation for the given period.

In at least one embodiment there is a computer device comprising program instructions stored on a non-transitory computer-readable medium, wherein the program instructions, when executed by a processor, cause the computer device to perform any of the methods above.

There is also provided a computer program comprising program instructions for causing a computer program to carry out the above method which may be embodied on a record medium, carrier signal or read-only memory.

### BRIEF DESCRIPTION OF THE DRAWINGS

The one or more embodiments of the invention will be more clearly understood from the following description of an embodiment thereof, given by way of example only, with reference to the accompanying drawings, in which:

FIG. 1 illustrates a flowchart 100 for an exemplary system, according to one or more embodiments of the invention;

FIG. 2 illustrates a conceptual chart 200 for an exemplary system, according to one or more embodiments of the invention; and

FIG. 3 illustrates a daily caseload service recommendation display 300 provided by an exemplary system, according to one or more embodiments of the invention.

### DETAILED DESCRIPTION OF THE INVENTION

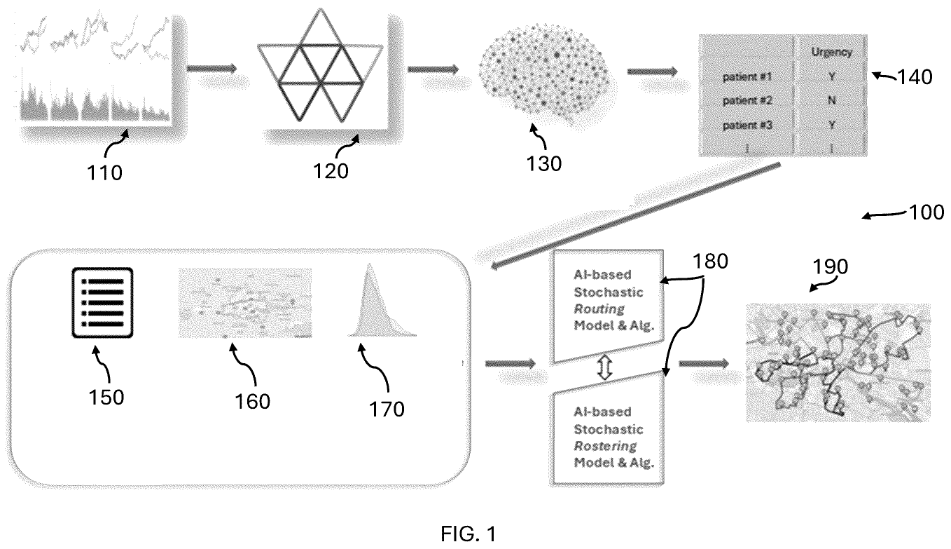

FIG. 1 illustrates a flowchart 100 for an exemplary system, according to one or more embodiments of the invention. This includes a dataset 110 consisting of patients' data points and time series. This dataset is the input to an optimisation algorithm 120 to convert the original dataset into a processable data table. The data table is then input to an ensemble machine learning algorithm 130 developed to triage patients. The output of this algorithm is a triage list 140 that is a sorted list of patients based on biographical and sociodemographic, and health attributes that are identified as the main determinants of a patient's stability level.

The organisational information 150 on patients, healthcare workers, and medical equipment includes: patient preferences and availability, patient medical needs, healthcare worker preferences and availabilities, healthcare workers specialities, and equipment availability.

The location specific data 160 includes: patient GPS information, distance information between a plurality of patients and healthcare workers, and traffic intensity distributions.

The above data 140, 150, 160, along with healthcare service length distributions 170 is input into the AI routing and rostering models 180, giving an output of the daily operational plan 190. This is the final recommendation of the system, which provides details for the tasks for all the healthcare workers, the order in which to complete them, and the routes they should take.

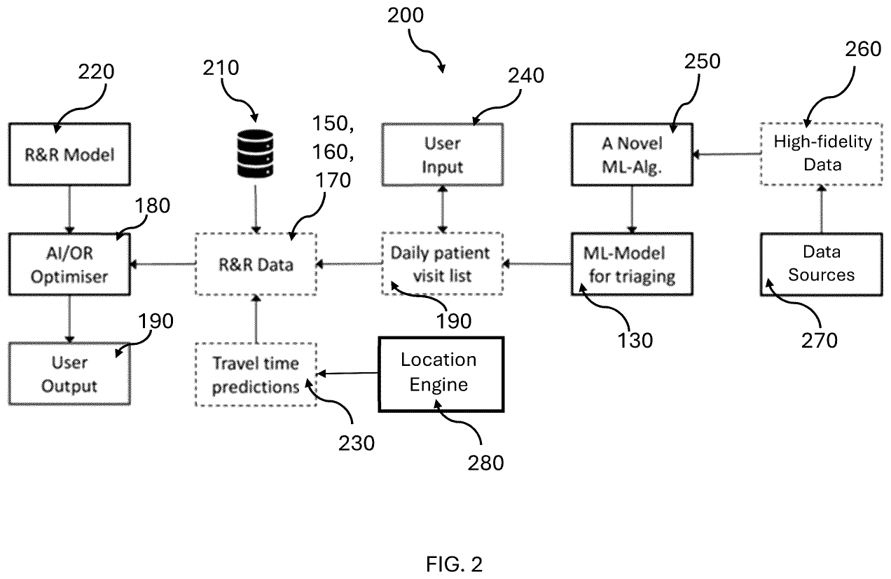

FIG. 2 illustrates a chart which includes the main components of a system, according to one or more embodiments of the invention.

The data sources 270 are used to generate high-fidelity data 260 for palliative care. The data sources can comprise a data generator or simulator. The generated high-fidelity data points 260 for patients are class labelled, where the categories represent the levels of urgency for patients, by expert healthcare decision makers. The data points can be time series data, for example feature vectors. The labelled high-fidelity data is used to train a predictive model 130 by a machine learning algorithm 250.

The resultant machine learning model (ML-Model for triaging) 130 is deployed to process the most up to date patient data and to generate a daily patient visit list 190. The user can then interact with this list, via user input 240, by viewing or altering the biographical and sociodemographic information or the health status for the patients.

The location engine 280 is designed specifically to collect travel times and other relevant data in a given location. Travel time predictions 230, which are not point forecasts, but in the form of probability distribution functions, are also recorded as part of the routing-and-rostering (R&R) data 150, 160, 170, as well as relevant information from a database 210.

The database 210 includes travel time prediction data for the location engine, which is stored in a tenant database as part of the data lake. This data can be retrieved from the Google Maps distance matrix API, for example, and includes the location ID, longitude, latitude, estimated time of travel, and estimated distance. R&R data 150, 160, 170 can also include information related to healthcare workers and patients, as well as the CNS daily patient visit list. Additional data can include the availability and preferences, of healthcare workers and patients, as well as the speciality of the workers and the daily patient visit list.

The daily patient visit list 190 comprises an ordered list of patients with respect to a set of social and healthcare measures. This may be presented to the healthcare decision maker to review and finalise the evaluation. At the end of this step, the daily patient caseload is generated and recorded as part of the R&R data 150, 160, 170 to be used for creating an instance of the R&R optimisation model 200, 180.

The collated R&R data 150, 160, 170 comprises information related to healthcare workers and patients, as well as the daily patient caseload and travel time distributions. An instance of the R&R model 220, which is a hybrid AI/operational research (OR) optimisation model 180, is created using the R&R data 150, 160, 170 for the given day to provide an output to a user 190.

The optimal (or a near-optimal) solution to the instance is obtained by using an AI/OR model optimiser 180. This solution fundamentally corresponds to a detailed recommended daily caseload plan involving scheduling and routing decisions 190. The daily caseload 190 presented to the healthcare operations manager can be revised in the final step of the resource optimisation process.

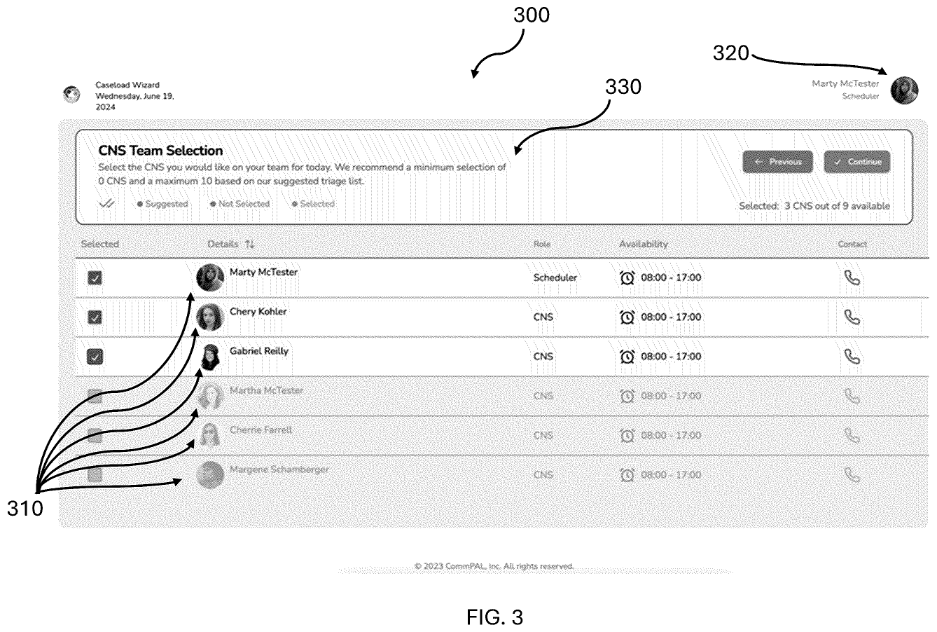

FIG. 3 illustrates a daily caseload service recommendation display 300 provided by an exemplary system comprising a user interface, according to one or more embodiments of the invention. This display 300 includes details of the user 320 and system participants 310. The system participants 310 can be Clinical Nurse Specialists (CNS), other healthcare workers, and admin staff. In addition, the display 300 can include prompts 330 to the user 320 enabling them to organise a daily caseload plan including scheduling and routing details. Through this interface, the user may reorganise inputs to the system, for example updating CNS availability and reorder patients throughout the day.

The one or more embodiments in the invention described with reference to the drawings comprise a computer apparatus and/or processes performed in a computer apparatus. However, the at least one embodiment of the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. a memory stick or hard disk. The carrier may be an electrical or optical signal which may be transmitted via an electrical or an optical cable or by radio or other means. The one or more embodiments of the invention may also include integration with existing platforms, such as an electronic health record or patient information management system. Furthermore, monitoring as a service (MaAS) functionality may be delivered via an API for the invention or through a customized web interface hosted on cloud platforms, thereby eliminating the requirement for on-premise deployment.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The at least one embodiment of the invention is not limited to the embodiments hereinbefore described but may be varied in both construction and detail.

1. A computer implemented resource optimisation system for community care comprising:
a predictive analytics component,
a prescriptive analytics component, and
a user interface component,
wherein,
the predictive analytics component is configured to receive a plurality of inputs relating to biographical and sociodemographic information and health status of a plurality of patients, and configured to output predictions for a plurality of future healthcare needs;
the prescriptive analytics component
receives a plurality of organisational data inputs related to patients information, and optionally one or more of healthcare workers information and medical equipment information for a given period,
which are combined with results from the predictive analytics component, and
outputs an optimal resource allocation for the given period following one or more guidelines; and
the user interface component is configured to provide a dynamic schedule which incorporates the optimal resource allocation that is outputted for the given period following said one or more guidelines using one or more interactive and visual representations.

2. The computer implemented resource optimisation system for community care of claim 1, wherein the plurality of inputs relating to the biographical and sociodemographic information of a plurality of patients comprises one or more of
an age of a patient,
degree of social support for said patient,
if said patient lives alone,
if the patient lives in residential care,
if the patient has a carer, and
if the patient has a family member as a carer.

3. The computer implemented resource optimisation system for community care of claim 1, wherein the predictive analytics component comprises a machine learning module adapted to assess patient care needs.

4. The computer implemented resource optimisation system for community care of claim 1, wherein the plurality of inputs relating to the health status of a plurality of patients comprises one or more of
patient diagnosis,
presence of multimorbidity in patient,
assessment of patient's breathing,
assessment of patient's sleeping,
assessment of patient's bowel movements,
assessment of need for urgent crisis event planning.

5. The computer implemented resource optimisation system for community care of claim 1, wherein one or more of the plurality of inputs relating to the health status of a patient are provided directly by a healthcare worker based on their assessment of the patient.

6. The computer implemented resource optimisation system for community care of claim 1, wherein the plurality of healthcare needs output by the predictive analytics component comprises a probability distribution to determine a need to implement one or more of medical interventions.

7. The computer implemented resource optimisation system for community care of claim 1, wherein the plurality of inputs related to patients contains one or more of
location of the patients, availability of the patients,
plurality of healthcare needs of the patients, and
health attributes of the patients output by the predictive analytics component.

8. The computer implemented resource optimisation system for community care of claim 1, wherein the plurality of organisational data inputs related to healthcare workers comprises one or more of location of the healthcare workers, availability of the healthcare workers, speciality or skillset of the healthcare workers.

9. The computer implemented resource optimisation system for community care of claim 1, wherein the plurality of interactive and visual representations comprising the dynamic schedule includes a daily operational plan, wherein the daily operational plan comprises a schedule for each healthcare worker with details of which patients the each healthcare worker will tend to, an order in which the each healthcare will the patients and recommended routes with which to travel between patient locations.

10. The computer implemented resource optimisation system for community care of claim 1, wherein the plurality of interactive and visual representations comprising the dynamic schedule display data relates to one or more of
stability of the plurality of patients' care plans and priorities,
available resources,
preferences of patients,
preference of healthcare workers,
projections of estimated time of arrival, travel distance, and
opportunity cost of reprioritising patients.

11. The computer implemented resource optimisation system for community care of claim 1, further comprising a preference component configured to manage healthcare worker preferences and patient preferences.

12. The computer implemented resource optimisation system for community care of claim 11, wherein
the preference component outputs preference constraints;
the plurality of organisational data inputs related to patients information and healthcare workers information, received by the prescriptive analytics component, comprises the preference constraints; and
the preference constraints comprise continuous care of a patient by at least one healthcare worker known to the patient.

13. The computer implemented resource optimisation system for community care of claim 1, further comprising a re-scheduler component configured to update the dynamic schedule provided by the user interface component.

14. The computer implemented resource optimisation system for community care of claim 13, wherein
the re-scheduler component is configured to process a plurality of real-time updated organisational data inputs related to at least one of patients information, healthcare workers information and medical equipment information, which is combined with results from the predictive analytics component; and
the dynamic schedule is updated when the re-scheduler determines the current dynamic schedule is not feasible, wherein determination by the re-scheduler is based on the plurality of real-time updated organisational data inputs.

15. The computer implemented resource optimisation system for community care of claim 9, further comprising a reporting component configured to store and report historical daily operational plans and data related to outcomes due to implementation of the daily operational plans, wherein the reporting component is configured to report one or more of
performance of implementation of the historical daily operational plans;
outcomes due to implementation of the historical daily operational plans;
historical user activities;
trends in the historical daily operational plans; and
greenhouse gas emissions caused by implementation of the historical daily operational plans.

16. The computer implemented resource optimisation system for community care of claim 9, further comprising an alert component configured to analyse the dynamic schedule for critical events and alert a user to the critical events via the user interface component, wherein the critical events comprise a risk of one or more of
high-priority patients not being tended to in a given time period;
a patients scheduled service being missed a second time;
patients not being seen within obligatory service level agreement window; and
a reschedule event occurring which is significantly less optimal than the previous schedule.

17. A computer implemented resource optimisation system for community care comprising:
a predictive analytics component and a prescriptive analytics component wherein,
the predictive analytics component is configured to receive a plurality of inputs relating to a health status of a plurality of patients, and configured to output predictions for a plurality of future healthcare needs;
the prescriptive analytics component
receives a plurality of organisational data inputs related to at least one of
patients information,
healthcare workers information, and
medical equipment information for a given period,
which are combined with results from the predictive analytics component, and
outputs an optimal resource allocation for the given period.

18. A computer-implemented method for resource optimisation for community care, comprising:
receiving, via a predictive analytics component, a plurality of inputs relating to biographical and sociodemographic information and health status of a plurality of patients, and outputting predictions for a plurality of future healthcare needs and health attributes;
receiving, via a prescriptive analytics component, a plurality of organizational data inputs related to at least one of patient information, healthcare worker information, and medical equipment information for a given period;
combining the plurality of organizational data inputs with the predictions from the predictive analytics component, and outputting an optimal resource allocation for the given period following one or more guidelines; and
providing, via a user interface component, a dynamic schedule incorporating the optimal resource allocation that is outputted for the given period, wherein the dynamic schedule is presented using one or more interactive and visual representations.

19. The computer-implemented method for resource optimisation for community care of claim 18, wherein
the plurality of inputs relating to the biographical and sociodemographic information of a plurality of patients comprises one or more of
an age of a patient,
degree of social support for the patient,
if the patient lives alone, if the patient lives in residential care,
if the patient has a carer, and
if the patient has a family member as a carer;
the plurality of inputs relating to the health status of a plurality of patients comprises one or more of
patient diagnosis,
presence of multimorbidity,
assessment of the patient's breathing, assessment of the patient's sleeping,
assessment of the patient's bowel movements, and
assessment of the need for urgent crisis event planning;
one or more of the plurality of inputs relating to the health status of a patient are provided directly by a healthcare worker based on an assessment by the healthcare worker of the patient; and
the plurality of inputs related to patients contains one or more of
location of the patients,
availability of the patients,
plurality of healthcare needs of the patients, and
health attributes of the patients output by the predictive analytics component, and
the plurality of organizational data inputs related to healthcare workers comprises one or more of
location of the healthcare workers,
availability of the healthcare workers, and
speciality or skillset of the healthcare workers.

20. The computer-implemented method for resource optimisation for community care of claim 18, wherein
the predictive analytics component comprises a machine learning module adapted to assess patient care needs; and
the plurality of interactive and visual representations comprising the dynamic schedule includes a daily operational plan, wherein the plan comprises a schedule for each healthcare worker with details of which patients the each healthcare worker will tend to, the order in which the each healthcare worker will visit the patients, and recommended routes with which to travel between patient locations.
